(19) United States
(12) Patent Application Publication
(10) Pub. No.: US 2019/0337989 A1
(43) Pub. Date: Nov. 7, 2019

(54) IMMUNITY INDUCER

(71) Applicants: RIKEN, Saitama (JP); Animal Allergy Clinical Laboratories Inc., Kanagawa (JP)

(72) Inventors: Kenichi MASUDA, Saitama (JP); Takashi SAITO, Saitama (JP); Yasuyuki ISHII, Saitama (JP); Ayato TAKADA, Hokkaido (JP); Manabu IGARASHI, Hokkaido (JP); Junki MARUYAMA, Hokkaido (JP); Yusuke SAITO, Kanagawa (JP); Takuya NARA, Kanagawa (JP)

(73) Assignees: RIKEN, Saitama (JP); Animal Allergy Clinical Laboratories Inc., Kanagawa (JP)

(21) Appl. No.: 16/345,599

(22) PCT Filed: Nov. 2, 2017
(86) PCT No.: PCT/JP2017/039752

§ 371 Date: Apr. 26, 2019

(30) Foreign Application Priority Data
Nov. 4, 2016 (JP) 2016-216753

(51) Int. Cl. C07K 14/005; A61K 39/145; A61K 39/39; A61P 31/16; C12N 7/00

(52) U.S. Cl. CPC C07K 14/005; A61K 39/145; A61K 2039/55572; A61P 31/16; C12N 7/00; A61K 39/39

(57) ABSTRACT

This application relates to an useful drug for prevention or treatment of avian influenza virus infection, and specifically provides a multiple antigen peptide comprising a dendritic core and 4-8 antigen peptides, wherein each of the antigen peptides is bound to a terminus of the dendritic core directly or through a spacer, and is a peptide consisting of 7-12 consecutive amino acids in the amino acid sequence of SEQ ID NO: 4, the multiple antigen peptide inducing the production of IgG antibodies in mammalian animals or birds, and further provides an immunity inducer comprising the multiple antigen peptide.

### TECHNICAL FIELD

The present invention relates to a multiple antigen peptide (MAP) for prevention or treatment of avian influenza virus infection, and to an immunity inducer comprising the MAP.

### BACKGROUND ART

The avian influenza is an infectious disease that develops when birds are infected with type A influenza viruses, and some of such viruses have acquired high virulence or high pathogenicity. In addition, because an avian influenza virus having potentials for human-to-human infection may induce a pandemic outbreak, the virus is regarded as hazardous. Avian influenza viruses are classified into subtypes in accordance with combinations of two types of glycoproteins on the virion surface; i.e., hemagglutinin (HA) and neuraminidase (NA). To date, 16 types of HAs (H1 to H16) and 9 types of NAs (N1 to N9) have been discovered. Examples of avian influenza virus subtypes include H1N1, H3N2, H5N1, H6N1, H7N7, H7N9, H9N2, and H10N8, and examples of subtypes that have caused bird-to-human infection include H7N7, H5N1, H7N3, H7N9, and H10N8. In particular, HA is susceptible to antibody-mediated selective pressure, thereby frequently causing antigenic variation.

Initial symptoms of the avian influenza virus infection include high fever, bronchial symptoms such as coughing, general malaise, and, occasionally, diarrhea, vomiting, abdominal pain, or the like, and it is further said that, when the infection becomes severe, the onset of pneumonia, ARDS, multiple organ failure, or the like occurs. Although neuraminidase inhibitors have been used as anti-influenza virus drugs for treatment of the infectious disease, there are opinions that their therapeutic effects are doubtful. Vaccines for infectious prevention have also been developed, however, their efficacy is not clear and thus the research and development of vaccines for human has been still continued.

As vaccines against avian influenza viruses, for example, Patent Literature 1 discloses liposomes comprising each peptide of GILGFVFTL (SEQ ID NO: 1), IILKANFSV (SEQ ID NO: 2), or GMFNMLSTV (SEQ ID NO: 3), bound thereto, and further discloses that mouse spleen cells pulsed with the peptide-bound liposome exhibited high CTL-induction activity and that, when mice immunized with the peptide (GILGFVFTL (SEQ ID NO: 1))-bound liposome were infected with the virus of subtype H1N1 or H3N2 via intranasal administration, the viral growth was suppressed significantly.

Moreover, Patent Literature 2 discloses general-use H5N1 vaccine compositions comprising H5 subtype hemagglutinin or antigen peptides thereof. This literature further discloses that an inactivated whole H5N1 virion vaccine, a mixture of 3 types of H5 hemagglutinin peptides, or the like was used alone or in combination with an adjuvant to induce high neutralizing antibody titers, and also that neutralizing epitopes were identified and, among them, a region in the sequence consisting of amino acids 138-218 in the mature HA1 region was determined as an antigenic epitope region comprising an important mutation.

In recent years, development of synthetic peptide vaccines has been intensively carried out. In particular, multiple antigen peptides (MAPs) are attracting attention. A MAP peptide can be obtained by using, as a core, a binding substance comprising a plurality of residues of lysine (Lys), which is one of amino acids, and, where needed, a cysteine residue (Cys), and peptides, which are corresponding to a part(s) of an antigen recognized by cells, bind to the α-amino and ε-amino groups of Lys, or to the sulfhydryl group of Cys, of the core.

For example, Patent Literature 3 discloses the use of MAP against Pneumococcus. Specifically, two sites in the antigen peptide of Pneumococcus are selected, and these two types of peptides are alternately arranged to construct an MAP-4 structure comprising 4 peptides in total. Furthermore, production of MAPs is disclosed in Patent Literature 4, Patent Literature 5, and Non-Patent Literatures 1 to 3.

### PRIOR ART LITERATURE

### Patent Literature

Patent Literature 1: JP 2011-126853 A
Patent Literature 2: JP Patent No. 5,815,676
Patent Literature 3: JP 2011-57691 A
Patent Literature 4: WO 1993/022343 A1
Patent Literature 5: WO 2015/190555 A1

### Non-Patent Literature

Non-Patent Literature 1: Myron Christodoulides and John E. Heckels, Microbiology 1994, 140: 2951-2960
Non-Patent Literature 2: Manju B. Joshi et al., Infection and Immunity 2001, 69: 4884-4890
Non-Patent Literature 3: Jon Oscherwitz et al., Infection and Immunity 2009, 77: 3380-3388

### SUMMARY OF INVENTION

### Technical Problem

As described in Background Art above, effective vaccines against avian influenza virus infection that can be used in clinical settings have not yet been found. Accordingly, there are demands on development of general-use vaccines against avian influenza virus that are effective for a plurality of types of the virus.

It is an object of the present invention to provide general-use immunity inducers for prevention or treatment of avian influenza virus infection using peptides derived from an avian influenza virus (e.g., vaccines).

### Solution to Problem

The present inventors have conducted intensive studies in order to attain the above object. As a result, the inventors have now found that particular regions in the amino acid sequences of HAs from various avian influenza viruses were more suitable as general-use antigen peptides, and have now developed multiple antigen peptides comprising a plurality of the antigen peptides, although the regions have not been reported to be capable of inducing antibody production. As a result, production of IgG antibodies to the peptides was observed, and surprisingly, it has been now found that effects of long-term production of IgM antibodies were induced, whereby the invention relating to immunity inducers that are useful as vaccines against avian influenza virus, was completed.

Specifically, the present invention has the following characterristics.

(1) A multiple antigen peptide comprising a dendritic core and 4-8 antigen peptides, wherein each of the antigen peptides is bound to a terminus of the dendritic core directly or through a spacer, and is a peptide consisting of 7-12 consecutive amino acids in the amino acid sequence of SEQ ID NO: 4, or a peptide which is the same as the peptide except that 1-3 amino acids are substituted.
(2) The multiple antigen peptide according to (1), wherein the peptide is a peptide consisting of 7-12 consecutive amino acids in the amino acid sequence of any of SEQ ID NOs: 5 to 9, or a peptide which is the same as the peptide except that 1-3 amino acids are substituted.
(3) The multiple antigen peptide according to (1), wherein the peptide is a peptide consisting of 7-12 consecutive amino acids in the amino acid sequence of SEQ ID NO: 15, or a peptide which is the same as the peptide except that 1-3 amino acids are substituted.
(4) The multiple antigen peptide according to (3), wherein the peptide is a peptide consisting of 7-12 consecutive amino acids in the amino acid sequence of any of SEQ ID NOs: 16 to 20, or a peptide which is the same as the peptide except that 1-3 amino acids are substituted.
(5) The multiple antigen peptide according to (4), wherein the peptide is a peptide consisting of 7-12 consecutive amino acids in the amino acid sequence of any of SEQ ID NOs: 21 to 25, or a peptide which is the same as the peptide except that 1-3 amino acids are substituted.
(6) The multiple antigen peptide according to any of (1) to (5), wherein all of the antigen peptides are peptides consisting of an identical amino acid sequence.
(7) The multiple antigen peptide according to any of (1) to (6), wherein the dendritic core comprises a plurality of lysine residues.
(8) The multiple antigen peptide according to (7), wherein the dendritic core further comprises a cysteine residue.
(9) The multiple antigen peptide according to any of (1) to (8), wherein the spacer comprises a polyoxyalkylene chain.
(10) The multiple antigen peptide according to any of (1) to (9), wherein the multiple antigen peptide is characterized by being represented by Formula (I):

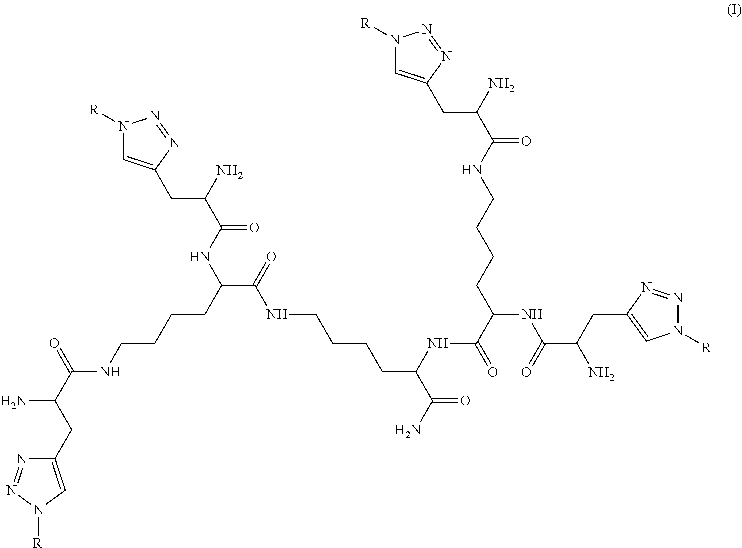

where R is:

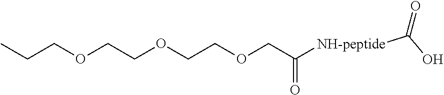

where the “peptide” represents an antigen peptide.
(11) An immunity inducer comprising, as active ingredients, one or at least two multiple antigen peptides according to any of (1) to (10).
(12) The immunity inducer according to (11), which further comprises an adjuvant having an ability to produce interferon γ.
(13) The immunity inducer according to (12), wherein the adjuvant is α-galactosylceramide or an analog thereof.
(14) The immunity inducer according to any of (11) to (13), which is used for treatment or prevention of avian influenza virus infection in a mammalian animal.
(15) The immunity inducer according to any of (11) to (14), which comprises a pharmaceutically acceptable carrier.
(16) A method for treatment or prevention of avian influenza virus infection in a mammalian animal, comprising administering the immunity inducer according to any of (11) to (15) to the mammalian animal.

According to the present invention, effects of inducing the immunity, such that MAP produced by allowing a peptide(s) in a particular region, which is from HAs of the avian influenza virus, to bind to a dendritic core can increase an IgG antibody titer and can function as a vaccine in a mammalian animal or a bird, are provided. Previously there have been a very small number of methods for effective prevention or treatment of avian influenza virus infection, but practical use of general-use vaccines against the avian influenza virus exerting effects on a plurality of types of the viruses has not yet been realized. Accordingly, the present invention is useful in terms of its advantageous effect.

This description includes all or part of the contents as disclosed in Japanese Patent Application No. 2016-216753, from which the present application claims priority.

### BRIEF DESCRIPTION OF DRAWINGS

FIG. 1 This figure shows MAP structures of MAP-2, MAP-4, MAP-8, and MAP-16.

FIG. 2 This figure shows the results of measurement of total IgG antibody titers against the avian influenza virus when the avian influenza MAP4 was administered intraperitoneally (ip) to mice. The total IgG antibody titers against the virus before administration, at 7 days after administration, at 14 days after administration, and at 21 days after administration were determined by measuring color development (A450) by ELISA using a peroxidase-labeled anti-IgG antibody as the secondary antibody and a peroxidase substrate.

FIG. 3 This figure shows the results of measurement of the subclass of IgG antibodies against the avian influenza virus when the avian influenza MAP4 was administered intraperitoneally (ip) to mice. The subclasses of the IgG antibodies against the virus before administration, at 7 days after administration, at 14 days after administration, and at 21 days after administration were determined by measuring color development (A450) by ELISA using a peroxidase-labeled anti-IgG antibody, a peroxidase-labeled anti-IgG2a antibody, or a peroxidase-labeled anti-IgG3 antibody as the secondary antibody and a peroxidase substrate.

FIG. 4 This figure shows the results of measurement of total IgM antibody titers against the avian influenza virus when the avian influenza MAP4 was administered intraperitoneally (ip) to mice. The IgM antibody titers against the virus before administration, at 7 days after administration, at 14 days after administration, and at 21 days after administration were determined by measuring color development (A450) by ELISA using a peroxidase-labeled anti-IgM antibody as the secondary antibody and a peroxidase substrate.

### MODES FOR CARRYING OUT THE INVENTION

The present invention is described in more detail.

### 1. Multiple Antigen Peptide

According to the first aspect, the present invention provides a multiple antigen peptide comprising a dendritic core and, as an antigen peptide, a peptide consisting of 7-12 continuous amino acids in the amino acid sequence of SEQ ID NO: 4 or a peptide which is the same as the peptide except that 1-3 amino acids are substituted, wherein the multiple antigen peptide comprises a same kind or different kinds of 4-8 antigen peptides, each of which has been bound to a terminus of the dendritic core directly or through a spacer, and induces the production of IgG antibodies in a mammalian animal.

The term “multiple antigen peptide” used herein refers to a macromolecular substance comprising a dendritic core having a dendritic polymer (i.e., dendrimer) structure and a plurality of a same kind or different kinds of peptides from avian influenza virus hemagglutinin (HA), each peptide being bound to a dendritic terminus of the core directly or through a spacer.

The term “a peptide which is the same as the peptide except that 1-3 amino acids are substituted” as used herein means a peptide in which the amino acid that substitutes for each of the 1-3 amino acids in the antigen peptide is any amino acid other than cysteine (Cys), preferably an amino acid having a chemical property (e.g., hydrophobicity, polarity, cationicity, anionicity, electric neutrality, or the like) or structural property (e.g., branch structure, aromaticity, or the like) similar to the substituted amino acid (i.e., the amino acid to be substituted).

The dendritic core is a dendritic supporting core for binding a plurality of, preferably 4-8, peptides (hereinafter, referred to as “antigen peptides” for convenience) from the avian influenza virus hemagglutinin. The dendritic core may have a commonly known structure, and a dendritic polymer basically having two or more identical branches that extend from a core molecule having at least two functional groups may be preferably selected. The dendritic core is also called dendritic polymer. Examples of the dendritic core include, but are not limited to, the structures described in U.S. Pat. Nos. 4,289,872 and 4,515,920. From the viewpoint of its simple production or the like, the dendritic core is preferably a peptide containing a plurality of lysine residues (K). The peptide containing lysine residues may also contain a cysteine residue (C). For example, in case of a K-K-K structure, which comprises three lysine residues (K), one molecule of the antigen peptide may be bound to each of the α-amino group side and the ε-amino group side of the lysine residue (K) at each terminus. In this case, at most 4 antigen peptides can be bound to the K-K-K structure. To the lysine residue (K), a spacer peptide may be bound through α-carboxyl group of the lysine residue. The spacer peptide is preferably a peptide consisting of 2-10 amino acid residues, such as K-K-C or K-βA-C, where βA represents a β-alanine residue and C represents a cysteine residue. When the amino acid residue at the N-terminus of the spacer peptide is, for example, a lysine residue (K), a K-K-K structure with at most 4 antigen peptides bound thereto as described above may be linked to the amino acid residue through α-amino group of the lysine residue. In such case, the prepared MAP has at most 8 antigen peptides.

According to the present invention, the antigen peptide is derived from the HA of the avian influenza virus.

The avian influenza virus has subtypes composed of many combinations of (H1 to H16)×(N1 to N9) as described above. Examples of subtypes include H1N1, H3N2, H5N1, H6N1, H7N7, H7N9, H9N2, and H10N8, and examples of subtypes recognized to cause bird-to-human infection include H7N7, H5N1, H7N3, H7N9, and H10N8.

The viral subtypes are distinguished from one another by difference in antigenicity of HA and NA. In the present invention, an extra attention was paid to HA, and antigen peptides for constructing the multiple antigen peptide were selected from regions whose amino acid sequences are highly conserved among subtypes.

The amino acid sequences of the antigen peptides thus selected consist of 7 to 12 continuous amino acids in the amino acid sequence as shown in SEQ ID NO: 4 below.

### SEQ ID NO: 4

### (Q or E)G(S, T, E, or V)G(Y, Q, T, or M)AAD(Q, L, Y, or R)(K, E, or D)STQ(N, A, K, or S)AID(G, Q, or K)(I or V)(T or N)(N, G, or S)

Examples of the amino acid sequence of SEQ ID NO: 4 above are the amino acid sequences of SEQ ID NOs: 5, 6, 7, 8, and 9, which are amino acid sequences corresponding to hemagglutinin of the subtypes H1N1, H3N2, H5N1, H7N9, and H9N2, respectively.

SEQ ID NO: 5
QGSGYAADQKSTQNAIDGITN
SEQ ID NO: 6
EGTGQAADLKSTQAAIDQING
SEQ ID NO: 7
QGSGYAADQESTQKAIDGVTN
SEQ ID NO: 8
QGEGTAADYKSTQSAIDQITG
SEQ ID NO: 9
QGVGMAADRDSTQKAIDKITS

Examples of amino acid sequences of HAs of the subtypes H1N1, H3N2, H5N1, H7N9, and H9N2 are as described below.

| (H1N1 HA) | |
|---|---|
| GenBank Accession NO: AGG82811 | |
| mat peptide 345 . . . 566 | |
| Influenza A virus (A/American black duck/New Brunswick/00326/2010 | |
| (H1N1)) | |
| SEQ ID NO: 10 | |
| 1 | MEAKLFVLFC TFTVLKADTI CVGYHANNST DTVDTVLEKN VTVTHSVNLL EDSHNGKLCS |
| 61 | LNGIAPLQLG KCNVAGWLLG NPECDLLLTA NSWSYIIETS NSENGTCYPG EFIDYEELRE |
| 121 | QLSSVSSFEK FEIFPKTNSW PNHETTKGVT AACSYSGASS FYRNLLWITK KGTSYPKLSK |
| 181 | SYTNNKGKEV LVLWGVHHPP TTSEQQSLYQ NTDAYVSVGS SKYNRRFTPE IAARPKVRGQ |
| 241 | AGRMNYYWTL LDQGDTITFE ATGNLIAPWY AFALNKGSDS GIITSDAPVH NCDTRCQTPH |
| 301 | GALNSSLPFQ NVHPITIGEC PKYVKSTKLR MATGLRNVPS IQSRGLFGAI AGFIEGGWTG |
| 361 | MIDGWYGYHH QNEQGSGYAA DQKSTQNAID GITNKVNSVI EKMNTQFTAM GKEFNNLERR |
| 421 | IENLNKKVDD GFLDVWTYNA ELLVLLENER TLDFHDSNVR NLYERVRSQL RNNAKELGNG |
| 481 | CFEFYHKCDD ECMESVKNGT YDYPKYSEES KLNREEIDGV KLESMGIYQI LAIYSTVASS |
| 541 | LVLLVSLGAI SFWMCSNGSL QCRICI |
| (H3N2 HA) | |
| GenBank Accession NO: AFY06393 | |
| mat_peptide 346 . . . 566 | |
| Influenza A virus (A/American black duck/New Brunswick/02650/2007 | |
| (H3N2)) | |
| SEQ ID NO: 11 | |
| 1 | MKTIIVLSCF FCLAFSQNPS ENNNNTATLC LGHHAVPNGT IVKTITDDQI EVTNATELVQ |
| 61 | SSSTGKICNN PHRILDGRDC TLMDALLGDP HCDVFQDETW DLYVERSSAF SNCYPYDVPD |
| 121 | YASLRSLVAS SGSLEFITEG FTWTGVTQNG GSGACKRGPA NGFFSRLNWL TKSGNAYPLL |
| 181 | NVTMPNNDDF DKLYVWGVHH PSTNQEQTSL YVQASGRVTV STRRSQQTII PNIGSRPWVR |
| 241 | GQSGRISIYW TIVKPGDILV INSNGNLIAP RGYFKMRTGK SSIMGSDAPV DTCISECITP |
| 301 | NGSIPNDKPF QNVNKITYGA CPKYVKQSTL KLATGMRNVP EKQTRGLFGA IAGFIENGWE |
| 361 | GMIDGWYGFR HQNSEGTGQA ADLKSTQAAI DQINGKLNRV IEKTNEKFHQ IEKEFSEVEG |
| 421 | RIQDLEKYVE DTKIDLWSYN AELLVALENQ HTIDLTDSEM NKLFEKTRRQ LRENAEDMGN |
| 481 | GCFKIYHKCD NACIESIRNG TYDHDIYRDE ALNNRFQIKG VELKSGYKDW ILWISFAISC |
| 541 | FLLCVVLLGF IMWACQRGNI RCNICI |
| (H5N1 HA) | |
| GenBank Accession NO: AAC32101 | |
| mat peptide 347 . . . 568 | |
| Influenza A virus (A/Chicken/Hong Kong/728/97 (H5N1)) | |
| SEQ ID NO: 12 | |
| 1 | MEKIVLLLAT VSLVKSDQIC IGYHANNSTE QVDTIMEKNV TVTHAQDILE RTHNGKLCDL |
| 61 | NGVKPLILRD CSVAGWLLGN PMCDEFINVP EWSYIVEKAS PANDLCYPGN FNDYEELKHL |
| 121 | LSRINHFEKI QIIPKSSWSN HDASSGVSSA CPYLGRSSFF RNVVWLIKKN SAYPTIKRSY |
| 181 | NNTNQEDLLV LWGIHHPNDA AEQTKLYQNP TTYISVGTST LNQRLVPEIA TRPEVNGQSG |
| 241 | RMEFFWTILK PNDAINFESN GNFIAPEYAY KIVKKGDSTI MKSELEYGNC NTKCQTPMGA |
| 301 | INSSMPFHNI HPLTIGECPK YVKSNRLVLA TGLRNTPQRE RRRKKRGLFG AIAGFIEGGW |
| 361 | QGMVDGWYGY HHSNEQGSGY AADQESTQKA IDGVTNKVNS IINKMNTPFE AVGREFNNLE |
| 421 | RRIENLNKKM EDGFLDVWTY NAELLVLMEN ERTLDFHDSN VKNLYDRVRL QLRDNAKELG |
| 481 | NGCFEFYHKC DNECMESVKN GTYDYPQYSE EARLNREEIS GVKLESMGTY QILSIYSTVA |
| 541 | SSLALAIMVA GLSLWMCSNG SLQCRICI |
| (H7N9 HA) | |
| GenBank Accession NO: AJJ95060 | |
| mat peptide 340 . . . 560 | |
| Influenza A virus (A/chicken/Dongguan/1022/2014 (H7N9)) | |
| SEQ ID NO: 13 | |
| 1 | MNTQILVFAL IAIIPTNADK ICLGHHAVSN GTKVNTLTER GVEVVNATET VERTNIPRIC |
| 61 | SKGKKTVDLG QCGLLGTITG PPQCDQFLEF SADLIIERRE GSDVCYPGKF VNEEALRQIL |
| 121 | RKSGGIDKEA MGFTYSGIRT NGATSACRRS GSSFYAEMKW LLSNTDNAAF PQMTKSYKNT |
| 181 | RKSPAIIVWG IHHSVSTAEQ TKLYGSGNKL VTVGSSNYQQ SFVPSPGARP QVNGLSGRID |
| 241 | FHWLMLNPND TVTFSFNGAF IAPDRASFLR GKSMGIQSGV QVDADCEGDC YHSGGTIISN |
| 301 | LPFQNIDSRA VGKCPRYVKQ RSLLLATGMK NVPEIPKGRG LFGAIAGFIE NGWEGLIDGW |
| 361 | YGFRHQNAQG EGTAADYKST QSAIDQITGK LNRLIEKTNQ QFELIDNEFN EVEKQIGNVI |
| 421 | NWTRDSITEV WSYNAELLVA MENQHTIDLA DSEMDKLYER VKRQLRENAE EDGTGCFEIF |
| 481 | HKCDDDCMAS IRNNTYDHSK YREEAMQNRI QIDPVKLSSG YKDVILWFSF GASCFILLAI |
| 541 | VMGLVFICVK NGNMRCTICI |
| (H9N2 HA) | |
| GenBank Accession NO: AMP44491 | |
| mat peptide 339 . . . 560 | |
| Influenza A virus (A/chicken/Anhui/36/2014 (H9N2)) | |
| SEQ ID NO: 14 | |
| 1 | METVSLITIL LVATVSNADK ICIGYQSTNS TETVDTLTEN NVPVTHAKEL LHTEHNGMLC |
| 61 | ATSLGQPLVL DTCTIEGLIY GNPSCDLSLE GREWSYIVER PSAVNGLCYP GNVENLEELR |
| 121 | SLFSSARSYQ RIQIFPDTIW NVSYDGTSSA CSGSFYKSMR WLTRKNGDYP IQDAQYTNNQ |
| 181 | GKNILFMWGI NHPPTDTTQR DLYTRTDTTT SVATEEINRI FKPLIGPRPL VNGLMGRIDY |
| 241 | YWSVLKPGQT LRIKSDGNLI APWYGHILSG ESHGRILKTD LKRGSCTVQC QTEKGGLNTT |
| 301 | LPFQNVSKYA FGNCSKYIGI KSLKLAVGLR NVPSRSSRGL FGAIAGFIEG GWSGLVAGWY |
| 361 | GFQHSNDQGV GMAADRDSTQ KAIDKITSKV NNIVDKMNKQ YEIIDHEFSE VETRLNMINN |
| 421 | KIDDQIQDIW AYNAELLVLL ENQKTLDEHD ANVNNLYNKV KRALGSNAVE DGKGCFELYH |
| 481 | KCDDQCMETI RNGTYNRRKY QEESKLERQK IEGVKLESEG TYKILTIYST VASSLVIAMG |
| 541 | FAAFLFWAMS NGSCRCNICI |

Preferable antigen peptides consist of 7-12 amino acids in the amino acid sequence of SEQ ID NO: 15, 16, 17, 18, 19, or 20 below.

SEQ ID NO: 15
AAD(Q, L, Y, or R)(K, E, or D)STQ(N, A, K, or
S)AID
SEQ ID NO: 16
AADQKSTQNAID
SEQ ID NO: 17
AADLKSTQAAID
SEQ ID NO: 18
AADQESTQKAID
SEQ ID NO: 19
AADKSTQSAID
SEQ ID NO: 20
AADRDSTQKAID

More preferable antigen peptides consist of the amino acid sequence of SEQ ID NO: 21, 22, 23, 24, or 25 below.

SEQ ID NO: 21
STQ(N, A, K, or S)AID
SEQ ID NO: 22
STQNAID
SEQ ID NO: 23
STQAAID
SEQ ID NO: 24
STQKAID
SEQ ID NO: 25
STQSAID

The antigen peptide according to the present invention can provide an immunity inducer that may be used as a vaccine against avian influenza virus infection, when a plurality of a same kind or different kinds of peptides, preferably a same kind of peptides, consisting of 7-12 continuous amino acids in the amino acid sequence of SEQ ID NO: 4, which was paid attention among HAs of the avian influenza virus, are bound to the above-described dendritic core.

The present invention also provides: an antigen peptide selected from the group consisting of peptides each consisting of 7-12 continuous amino acids in the amino acid sequence as shown in any of SEQ ID NOs: 5 to 9; an antigen peptide selected from the group consisting of peptides each consisting of 7-12 continuous amino acids in the amino acid sequence as shown in any of SEQ ID NO: 15 to 25; and an antigen peptide selected from the group consisting of peptides each consisting of any of the amino acid sequences as shown in SEQ ID NOs: 22 to 25.

The antigen peptides constituting the multiple antigen peptide (MAP) of the present invention are bound to the termini of the dendritic core directly or through a spacer, wherein preferably the antigen peptides are covalently bound to each terminus of the dendritic core one by one. For example, a functionalized dendritic core may be bound to a functionalized solid-phase resin, and a reactive functional group of each antigen peptide may be reacted with and bound to a reactive functional group at the dendritic terminus (W. Kowalczyk et al., J. Pep. Sci. 2011, 17: 247-251). In this case, the antigen peptides may be synthesized by known techniques including synthesizing by use of an automated peptide synthesizer based on predetermined amino acid sequences (for example, J. M. Stewart and J. D. Young, Solid Phase Peptide Synthesis, 2^{nd} ed., Pierce Chemical Company, 1984; and G. B. Fields et al., Principles and Practice of Peptide Synthesis, in G. A. Grant (ed.): Synthetic Peptides: A User's Guide, W.H. Freeman, 1992). Alternatively, the antigen peptides may be prepared by using known DNA recombination techniques (for example, M. R. Green and J. Sambrook, Molecular Cloning A Laboratory Manual, Vol. 1 and Vol. 2, Cold Spring Horbor Laboratory Press, fourth edition, 2012).

The MAP of the present invention comprises a plurality of, preferably 2-16 and more preferably 4-8, antigen peptides, and the antigen peptides may be of a same kind or different kinds, preferably of a same kind. As used herein, the term “a same kind of antigen peptides” means a peptide having the same epitope properties, which peptide includes a peptide having a high identity. The “peptide having a high identity” is a peptide having substitution of 1-3 amino acids, preferably 1 or 2 amino acids, more preferably a single (1) amino acid, relative to any one of a plurality of antigen peptides. The amino acid(s) that substitutes an amino acid(s) in the antigen peptide is any amino acid except for cysteine (Cys), preferably an amino acid that has a similar chemical property (e.g., hydrophobicity, polarity, cationicity, anionicity, electric neutrality, or the like) or a structural property (e.g., branch structure, aromaticity, or the like) to the substituted amino acid. As used herein, the term “same epitope properties” means properties that can induce in vivo production of an IgG antibody capable of binding to a target protein or polypeptide of interest and of inducing immunity against the virus. When different kinds (that is, not “a same kind”) of antigen peptides are used, at least one of each different antigen peptide is bound to the dendritic core.

Examples of “subject to which the multiple antigen peptide is administered” herein (hereafter, referred to as “subject” for convenience) include mammalian animals and birds (or avian), such as humans, livestock animals (e.g., cows, pigs, sheeps, goats, and camels), fowls (e.g., chickens, ducks, and quails), pet animals, (e.g., dogs, cats, and birds), racing animals (e.g., horses and chickens), and ornamental animals raised in zoos, humans being preferable.

The MAP of the present invention induces production of class-switched antibodies in the body of a subject. The antibodies produced in the present invention are IgG, IgA, or IgE, preferably IgG. In general, when a foreign substance invades into the body, IgM antibody is produced from B2 B cells within about one week to allow initial protection to function in the body. Since IgM, however, has a short half-life, its antibody titer in blood decreases within about 1 week to 10 days. Following the production of IgM, activation of T cells reactive to the foreign substance occurs gradually in the body, resulting in producing IgG antibodies so as to enhance protection by humoral immunity. Once IgG antibodies are produced, because their half-lives are long, their antibody titers in blood are maintained over a period of from several weeks to several months or longer.

In another aspect, the MAP of the present invention is capable of stimulating B cells in the innate immune system (B1 B cells) to cause production of IgM for a longer period compared to the production by B2 B cells. IgM increased in blood by administration of the MAP of the present invention is confirmed for, for example, 14 days or longer, preferably 21 days or longer.

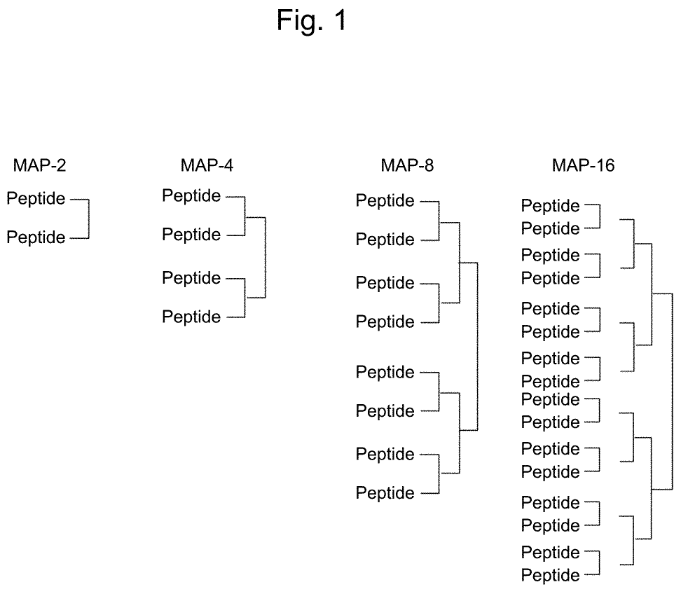

The MAP of the present invention has, for example, the structure shown in FIG. 1. In particular, the MAP has a dendritic structure comprising 4-8 antigen peptides, preferably the same antigen peptides, as shown as MAP-4 or MAP-8. Specifically, the MAP may have the MAP-4 structure of the following Formula (I), but is not limited to thereto.

MAP represented by Formula (I):

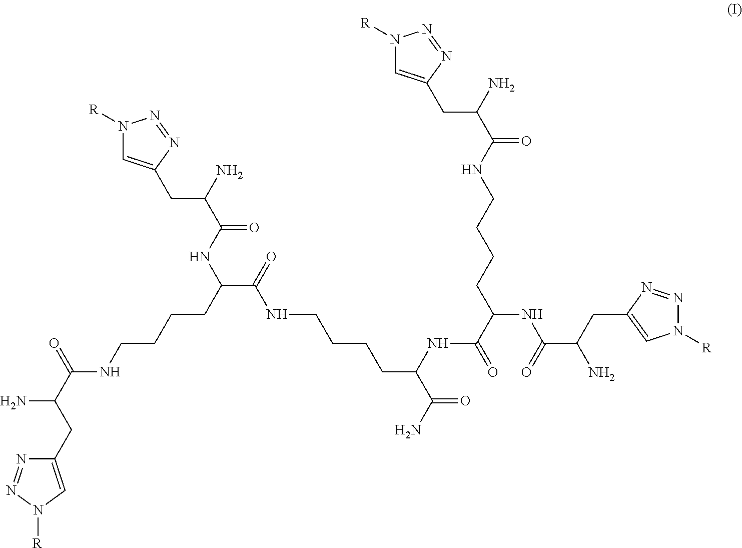

where R is:

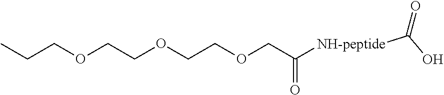

where the “peptide” represents an antigen peptide.

### 2. Production of Multiple Antigen Peptide (MAP)

The MAP of the present invention can be prepared by a method comprising, for example, the following steps of:

(1) providing a dendritic core having reactive functional groups;

(2) providing a plurality of a same kind or different kinds of antigen peptides each having a reactive functional group;

(3) producing a multiple antigen peptide by reaction of binding the, reactive functional group of the dendritic core to the reactive functional group of each antigen peptide; and

(4) collecting the multiple antigen peptide.

As described above, the dendritic core is a dendritic supporting core to bind a plurality of a same kind or different kinds, preferably a same kind, of the antigen peptides, preferably a same kind of (preferably, identical) 4-8 antigen peptides. The dendritic core may have a commonly known structure. The dendritic core preferably comprises a plurality of lysine residues (K), and may also contain a cysteine residue (C). As shown in the exemplified structures of the MAP of the invention in FIG. 1 (preferably, a structure such as MAP-4 or MAP-8), the portions other than the 4-8 antigen peptides are formed by the dendritic core. The dendritic core preferably comprises, for example, a K-K-K sequence for MAP-4, or comprises, for example, a K-K-K-K-K sequence for MAP-8. Usually, a spacer peptide is bound to the K in the center of these sequences. Preferably, the spacer peptide is a peptide consisting of two or more amino acid residues, such as K-K-C or K-βA-C(where βA represents a β-alanine residue), but is not limited to them. The K or K-K in each of the left and right, other than the K in the center, is designed such that two antigen peptides are bound per one K. A spacer may be arranged between the dendritic core and each peptide. The spacer is preferably a highly hydrophilic group containing a polyoxyalkylene chain (e.g., polyoxyethylene chain or polyoxypropylene chain). The number of repeats of oxyalkylene units in the polyoxyalkylene chain is 2 or more, preferably 2-50, more preferably 3-30.

Each terminus of the dendritic core may have an appropriate functional group for binding to the antigen peptide. The functional group is not limited as long as it can be used for modification of a protein, examples of which include amino group, sulfhydryl group, acetylene group, and N-hydroxysuccinimidyl group.

On the other hand, the functional group in the antigen-peptide side is any functional group that is capable of undergoing binding reaction with a terminal functional group of the dendritic core. Examples of this functional group include an N-hydroxysuccinimidyl group for amino group, a sulfhydryl group or carboxyl group for sulfhydryl group, and an azide group for acetylene group. The antigen peptide is as described above.

According to an embodiment of the present invention, the dendritic core having the K-K-K sequence has the structure shown below:

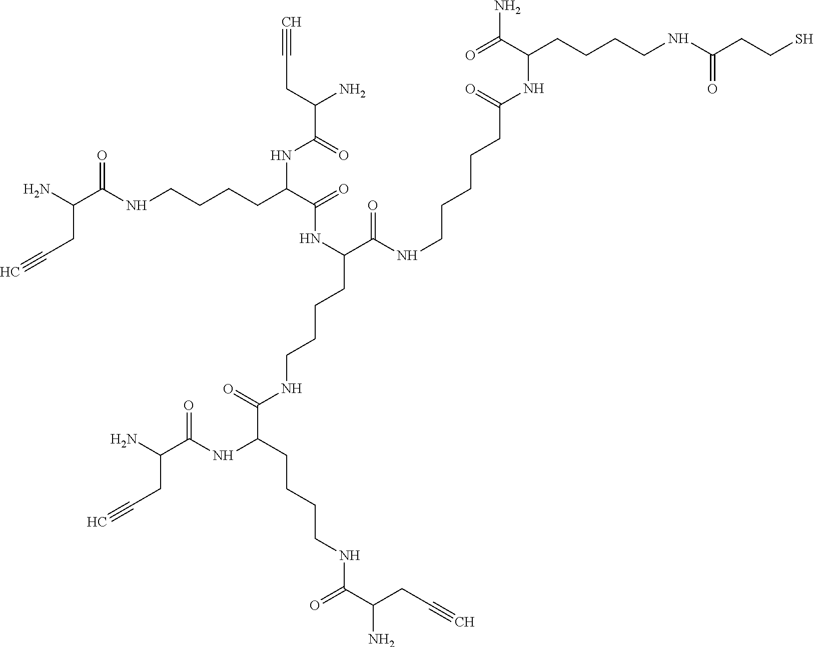 [SMILES: C#CCC(N)C(=O)NCCCCC(NC(=O)C(N)CC#C)C(=O)NCCCCC(NC(=O)C(CCCCNC(=O)C(N)CC#C)NC(=O)C(N)CC#C)C(=O)NCCCCCC(=O)NC(CCCCNC(=O)CCS)C(N)=O]

wherein each terminal functional group has an acetylene group.

The terminal functional group of the antigen peptide reacting with the acetylene group in the above structure is an azide group. In this case, binding takes place in accordance with the Huisgen reaction shown below. In the formula below, R₁ represents a dendritic core portion, and R₂ represents an antigen peptide.

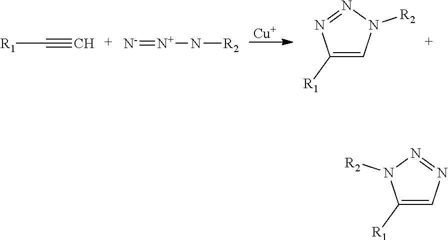

This reaction is a reaction in which an alkyne is bound to an azide by using a monovalent copper ion as a catalyst, and the reaction product is said to be stable and to hardly show side reactions. This reaction is thus attracting attention as the click chemistry. The copper ion catalyst solution can be prepared using an aqueous copper sulfate pentahydrate solution and ascorbic acid.

In the step of collecting MAP, the peptide is purified. The peptide may be collected by general methods of purification of proteins or polypeptides. Examples of such techniques include gel filtration chromatography, ion-exchange chromatography, hydrophobic interactive chromatography, reverse-phase chromatography, affinity chromatography, and high-performance liquid chromatography (HPLC), and such techniques can be performed alone or in combination. The target products can be identified by, for example, nuclear magnetic resonance (NMR) spectroscopy, mass spectrometry, LC/MS, amino acid analysis, and the like.

### 3. Immunity Inducer

The present invention provides an immunity inducer, which further comprises one or at least two multiple antigen peptides (MAPs) as described above. The immunity inducer of the present invention is a pharmaceutical preparation that induces production of IgG antibodies, or IgG and IgM antibodies.

The immunity inducer according to the present invention induces production of IgG antibody against avian influenza virus infection. Thus, the immunity inducer can be used as a pharmaceutical composition for prevention, treatment, or amelioration of the infectious disease, or can be used as a vaccine.

The immune inducer of the present invention, as a pharmaceutical composition, can sustain production of IgM antibody against avian influenza virus infection for a long period, and also enables prevention of the infection in uninfected individuals. In addition, by production of IgM antibody for a long period, the immune inducer can be used for prevention of transmission of the infection from infected individuals to uninfected individuals.

The effective dose of the MAP of the present invention per administration in humans is, but is not limited to, for example about 0.05-2.5 μg/kg body weight to 1-10 mg/kg body weight for MAP-4, or, for example 0.5-25.0 μg/kg body weight to 1-10 mg/kg body weight for MAP-8. Herein, the dose may be appropriately changed depending on body weights, ages, sexes, symptoms, severities, administration methods, and the like, of subjects including human.

The immune inducer of the present invention may be in the form of, for example, solutions, suspensions, tablets, injection solutions, granules, emulsions, nebulas, or the like, and may appropriately contain an additive(s) such as vehicle, diluent, binder, disintegrator, lubricant, solubilizer, preservative, flavor, surfactant, and the like. An Adjuvant is basically not needed as long as production of interferon γ can be seen in a subject that the immune inducer is administered to, but it may be added where needed.

The immune inducer of the present invention may comprise an adjuvant. The adjuvant is appropriately selected depending on isotypes of desired antibodies. For example, where IgG is preferentially produced, the adjuvant is a substance that predominantly induces production of interferon γ. The substance that induces production of interferon γ includes, but is not limited to, for example α-galactosylceramide, α-galactosylceramide analogs, and bacterial oligonucleotide CpG. Examples of the α-galactosylceramide analogs include, but are not limited to, compounds described in WO 2007/099999 (U.S. Pat. No. 8,163,705 B), WO 2009/119692 (U.S. Pat. No. 8,551,959 B), WO 2008/102888 (U.S. Pat. No. 8,299,223 B), WO 2010/030012 (U.S. Pat. No. 8,580,751 B), WO 2011/096536 (U.S. Pat. No. 8,853,173 B), and WO 2013/162016 (US 2015-0152128 A). In addition, similarly to the adjuvant, interferon γ may be included for enhancement of the effect of an IgG antibody induction by the immune inducer of the present invention.

The immunity inducer according to the present invention can be used for prevention, prevention of spread, or treatment of avian influenza virus infection, as a pharmaceutical composition.

Thus, the present invention further provides a method for prevention or treatment of the disease described above, comprising administering the MAP or the immunity inducer to a subject. In this method, the antibody production encompasses IgG antibody production and IgM antibody production. In the method of the present invention, the antibody production can be performed for treatment, prevention, or prevention of spread of avian influenza virus infection.

Examples of administration routes include, but are not limited to, intravenous administration, intraarterial administration, intranasal administration, transmucosal administration, intraperitoneal administration, intrarectal administration, hypodermic administration, intramuscular administration, and oral administration.

The immunity inducer according to the present invention may be prepared by further containing a pharmaceutically acceptable carrier.

The term “pharmaceutically acceptable” has a meaning commonly used in the pharmaceutical industry, and indicates, in some cases, that a substance, composition or the like can be used without causing allergic reactions or similar harmful reactions when it is administered to humans. Preparation of an aqueous composition comprising a protein as the active ingredient is sufficiently understood in the art. Such a composition may be prepared typically as an injection solution, liquid solution, or suspension, and may also be prepared as a solid formulation suitable for dissolution or suspension in the liquid before injection. The prepared product may also be emulsified.

The “carrier” includes any or all solvents, dispersion media, vehicles, coatings, diluents, antibacterial and antifungal agents, isotonic and absorption delaying agents, buffers, carrier solutions, suspensions, and colloids. Examples of the carrier include: buffers of phosphate, citrate, and other organic acid salts; antioxidants containing ascorbic acid; low molecular weight polypeptides (less than about 10 amino acid residues); proteins (for example, serum albumin, gelatin, or immunoglobulins); hydrophobic polymers (for example, polyvinylpyrrolidone); amino acids (for example, glycine, glutamine, asparagine, arginine, or lysine); monosaccharides, disaccharides, and other carbohydrates such as glucose, mannose, or dextran; chelating agents such as EDTA; sugar alcohols such as mannitol or sorbitol; salt-forming counter ions such as sodium; and/or nonionic surfactants (for example, polyoxyalkylene-based surfactants). Use of the media and substances for pharmaceutically active substances is well known in the art. It is expected that any conventional medium or substance can be used in therapeutic compositions as long as the medium or substance is not incompatible as an active ingredient. An auxiliary active ingredient may also be incorporated in the composition.

For the immune inducer of the present invention, various surfactants used for preparation may be used. The types of the surfactants include, but are not limited to, nonionic surfactants, cationic surfactants, anionic surfactants, and amphoteric surfactants, preferablynonionic surfactants. Examples of the nonionic surfactants include: polyoxyalkylene-based nonionic surfactants such as polyoxyethylene monoalkyl ethers or polyoxyethylene monoaryl ethers; higher fatty acid esters of polyols (for example, sorbitan and sorbitol); and products prepared by addition of ethylene oxide to higher fatty acid esters of polyols by polymerization.

The immune inducer of the present invention may further comprise one or more additional components. Examples of the additional components include, but are not limited to, suspending agents, stabilizers, and dispersants. For stabilization of the immune inducer of the invention, the isoelectric point of the MAP may be lowered to improve its metabolic stability. Specifically, an acidic amino acid(s) (for example, asparagine or glutamic acid) and/or a deoxynucleotide(s) (for example, GpC oligonucleotide or CpG oligonucleotide) may be further contained in the immune inducer. In one embodiment, an acidic amino acid(s) and/or a deoxyoligonucleotide(s) may be directly bound to the MAP of the present invention.

### EXAMPLES

The present invention will be described in more detail with reference to the following examples, although the technical scope of the t invention is not limited to these examples.

### Example 1

### <Structure of Multiple Antigen Peptide (MAP)>

The MAP structure represented by Formula (I) below was used:

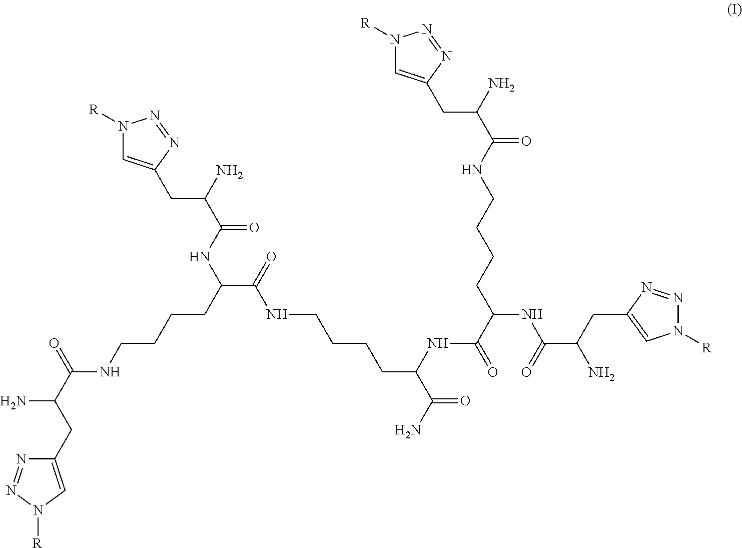

where R is:

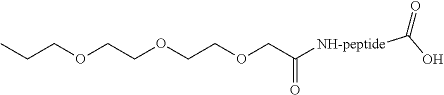

where the “peptide” represents an antigen peptide.

A peptide derived from avian influenza virus hemagglutinin; i.e., STQKAID (SEQ ID NO: 24), was selected and used as an antigen peptide for preparation of MAP.

### <Synthesis of Avian Influenza MAP4>

### 1. List of Abbreviations

### NH2-SAL-Trt(2-Cl)-Resin: Rink-Bernatowitz-amide Barlos Resin (Watanabe Chemical Industries, Ltd., Hiroshima, Japan)

Fmoc-Lys(Fmoc)-OH: N-α,N-ε-bis(9-fluorenylmethoxycarbonyl)-L-lysine (Watanabe Chemical Industries, Ltd.)
Boc-Pra-OH: N-Boc-L-propargylglycine (Tokyo Chemical Industry Co., Ltd., Tokyo, Japan) N₃-PEG-COOH: 11-Azido-3,6,9-trioxaundecanoic acid (Tokyo Chemical Industry Co., Ltd.)
H-Asp(OtBu)-Trt(2-Cl) resin: L-aspartic acid β-t-butyl ester 2-chlorotrityl resin (Watanabe Chemical Industries, Ltd.)
Fmoc-Ala-OH: N-α-(9-fluorenylmethoxycarbonyl)-L-alanine (Watanabe Chemical Industries, Ltd.)

### Fmoc-Ieu-OH: N-α-(9-Fluorenylmethoxycarbonyl)-L-isoleucine (Watanabe Chemical Industries, Ltd.)

Fmoc-Gln(Trt)-OH: N-α-(9-fluorenylmethoxycarbonyl)-N-β-trityl-L-glutamine (Watanabe Chemical Industries, Ltd.)
Fmoc-Ser(tBu)-OH: N-α-(9-fluorenylmethoxycarbonyl)-O-(t-butyl)-L-serine (Watanabe Chemical Industries, Ltd.)
Fmoc-Thr(tBu)-OH: N-α-(9-fluorenylmethoxycarbonyl)-O-(t-butyl)-L-threonine (Watanabe Chemical Industries, Ltd.)
HATU: O-(7-aza-1H-benzotriazol-1-yl)-N,N,N′,N′-tetramethyluronium luorophosphate (Genscript)
DIEA: N,N-diisopropylethylamine (Wako Pure Chemical Industries, Ltd., Osaka, Japan, for peptide synthesis)
DMF: N,N-dimethylformamide (Kanto Chemical Co., Inc., Tokyo, Japan, for peptide synthesis)
TFA: 2,2,2-Trifluoroacetic acid (Wako Pure Chemical Industries, Ltd.)

### TIPS: Triisopropylsilane (Watanabe Chemical Industries, Ltd.)

### Thioanisole (Watanabe Chemical Industries, Ltd.)

m-Cresol (Tokyo Chemical Industry Co., Ltd.)

### DCM: Dichloromethane (Kanto Chemical Co., Inc.)

ACN: acetonitrile (Kanto Chemical Co., Inc., for HPLC)
α-CHCA: α-cyano-4-hydroxycinnamic acid
Preparative column: YMC-Pack Pro C18, 20 mm (I.D.)×250 mm (length); particle diameter: 5 μm, pore diameter: 12 m (YMC)
Analysis column: YMC-Pack Pro C18, 4.6 mm (I.D.)×250 mm (length), particle diameter: 5 μm; pore diameter: 12 μm (YMC)

### MALDI-TOF MASS: Matrix Assisted Laser Desorption Ionization Time Of Flight Mass Spectrometry

D.W.: distilled water
Copper sulfate pentahydrate (Kanto Chemical Co., Inc.)
Ascorbic acid (Kanto Chemical Co., Inc.)

### 2. Synthesis of MAP Core

MAP4 is used as an example to describe a method of synthesis of an MAP core below. MAP core synthesis was carried out in accordance with the general technique of Fmoc solid-phase synthesis, and all steps were manually carried out. Specifically, synthesis was carried out using 1 mmol NH2-SAL-Trt(2-Cl)-Resin as a solid-phase carrier by the procedures described below.

TABLE 1

| | | Amino acid | Reaction time | Number |
|---|---|---|---|---|
| | Step | (mmol) | (min) | of times |
| 1. | Deblock | — | 7 | 1 |
| 2. | Fmoc-Lys(Fmoc)-OH | 3 | 15 | 1 |
| 3. | Deblock | — | 7 | 1 |
| 4. | Fmoc-Lys(Fmoc)-OH | 3 | 15 | 2 |
| 5. | Deblock | — | 7 | 2 |
| 6. | Boc-Pra-OH | 4 | 15 | 1 |
| 7. | Boc-Pra-OH | 2 | 15 | 1 |

* Upon reaction, the mixture was gently stirred using a reciprocating shaker
* After a step was completed, solid phase was sufficiently washed with DMF and then moved to the next step
* Deblock refers to a step of deprotecting the N-terminal Fmoc group with a 20% piperidine/DMF solution
* Coupling of each amino acid was carried out at the following composition ratio (molar ratio):
* Protected amino acid:HATU:DIEA = 1:1:2
* The reagents were dissolved in DMF such that the amino acid solution concentration during reaction was 0.2M.

After the completion of synthesis, D.W. (ml):TIPS (ml):TFA (ml)=1.5:1.5:30 was added to 0.1 mmol of the solid-phase, and the mixture was stirred for 1.5 hours, followed by cleavage and deprotection. After the cleavage, the solution was collected by filtration and then concentrated under reduced pressure, and a small amount of water was added thereto, followed by freeze-drying. Thereafter, the reaction product was purified by reversed-phase HPLC using 0.1% TFA (trifluoroacetic acid) and ACN (acetonitrile) as an eluate. The purified product was subjected to mass analysis using MALDI-TOF MASS to identify the target product.

### Purification Conditions:

Eluate A: 0.1% TFA; Eluate B: 0.1% TFA in ACN solution

Equilibration: 100% Eluate A, 10 ml/min, 10 minutes

Elution: Linear gradient of 100% Eluate A→70% Eluate A/30% Eluate B, 10 ml/min, 30 minutes

### Mass Analysis:

Matrix solution: 0.1% TFA in 50% aqueous ACN solution containing 10 mg/ml α-CHCA

Sample: HPLC eluate or 0.1% TFA in 50% aqueous ACN solution (approximately 1 mg/ml peptide)

The matrix solution is mixed with the sample at 1:1 to form a mixed crystal on the plate.

### 3. Synthesis of Antigen Peptide

As with the case of MAP core synthesis, antigen peptide synthesis was carried out in accordance with the method of Fmoc solid-phase synthesis. Specifically, synthesis was carried out using 1 mmol H-Asp(OtBu)-Trt(2-Cl) resin as a solid-phase carrier by the procedures described below.

The antigen peptide sequence was N3-PEG-STQKAID-OH, and the peptide was extended from the C terminus toward the N terminus.

TABLE 2

| | | Amino acid | Reaction time | Number |
|---|---|---|---|---|
| Step | | (mmol) | (min) | of times |
| 1. | Fmoc-Amino acid | 3 | 15 | 1 |
| 2. | Step 1 and Step 2, where the amino acid(s) was/were changed | | | |
| | in accordance with the sequence, are repeated. | | | |
| 3. | Deblock | — | 7 | 1 |
| 4. | N₃-PEG-COOH | 1.2 | 15 | 1 |

* After a step was completed, solid phase was sufficiently washed with DMF and then moved to the next step
* Upon reaction, the mixture was gently stirred using a reciprocating shaker
* Deblock refers to a step of deprotecting the N-terminal Fmoc group with a 20% piperidine/DMF solution
* Fmoc-amino acids used herein are as follows: Fmoc-Ala-OH, Fmoc-leu-OH, Fmoc-Gln(Trt)-OH, Fmoc-Ser(tBu)-OH, Fmoc-Thr(tBu)-OH
* The amino acids were coupled with each other in the following composition ratio (molar ratio): Protected amino acid (mmol):HATU (mmol):DIEA (mmol):DMF (ml) = 3:3:6:12 ml

After the completion of synthesis, thioanisole (ml):m-cresol (ml):TIPS (ml):TFA (ml) (5.4:1.5:0.9:30) was added to 1 mmol of the solid-phase, and the mixture was stirred for 1.5 hours, followed by cleavage and deprotection. After the cleavage, the solution was collected by filtration and then concentrated under reduced pressure. In addition, ether was added thereto, and the precipitate was collected to obtain an unpurified peptide. The unpurified peptide was purified by reversed-phase HPLC using 0.1% TFA in ACN as an eluate.

The purified product was subjected to mass analysis using MALDI-TOF MASS to identify the target product.

### Purification Conditions:

Eluate A: 0.1% TFA; Eluate B: 0.1% TFA in ACN solution

Equilibration: 90% Eluate A/10% Eluate B, 10 ml/min, 10 minutes

Elution: Linear gradient of 90% Eluate A/10% Eluate B→60% Eluate A/40% Eluate B, 10 ml/min, 30 minutes

### HPLC Analysis Conditions:

Eluate A: 0.1% TFA; Eluate B: 0.1% TFA in ACN

Equilibration: 95% Eluate A/5% Eluate B, 10 ml/min, 10 minutes

Elution: Linear gradient of 95% Eluate A/5% Eluate B→60% Eluate A/40% Eluate B, 10 ml/min, 30 minutes

### Mass Analysis: As Described Above.

### 4. Synthesis of MAP

The MAP core was allowed to bind to the antigen peptides by the Huisgen reaction. Specifically, the alkyne in the MAP core was activated with the aid of Cu⁺ and allowed to react with the azide group at the N terminus of each antigen peptide, thereby leading to binding them through triazole.

### (Step 1)

The MAP core and the antigen peptide were dissolved in an aqueous solution of 0.1% TFA. In this case, the mixing ratio was the MAP core 28 mg (36 μmol): the antigen peptide 145 mg (148 μmol), and they was dissolved in 2 ml of an aqueous solution of 0.1% TFA (a peptide solution).

### (Step 2)

An aqueous solution of copper sulfate pentahydrate and an aqueous solution of ascorbic acid were prepared in the manner described below. Copper sulfate pentahydrate 50 mg (200 μmol) was dissolved in 1 ml of D.W. (an aqueous solution of copper sulfate). Also, ascorbic acid 176 mg (1 mmol) was dissolved in 1 ml of D.W. (an aqueous solution of ascorbic acid). Subsequently, the total amount of the aqueous solution of copper sulfate was mixed with the total amount of the aqueous solution of ascorbic acid (a Cu⁺ solution).

### (Step 3)

Subsequently, the Huisgen reaction was carried out. Specifically, 2 ml of the peptide solution was mixed with 1.1 ml of the Cu⁺ solution, and the mixture was subjected to the reaction at room temperature for several hours.

### (Step 4)

The target product was purified by reversed-phase HPLC using 0.1% TFA in ACN as an eluate, and the resultant was then freeze-dried.

Mass analysis was conducted using MALDI-TOF MASS to identify the target product.

The purification conditions were the same as those concerning the antigen peptide described above.

The HPLC purity test was carried out under the same conditions as described concerning the antigen peptide.

Mass analysis was carried out in the same manner as described above.

The results of synthesis were as described below.

TABLE 3
MAP core

| | | Theoretical | | Yield | | Percent | Molecular | |
|---|---|---|---|---|---|---|---|---|
| Name | Sequence | mg | μmol | mg | μmol | yield (%) | weight | Measured |
| MAP4 Core | Pra₄-K₂-K-NH₂ | 782 | 1000 | 501 | 641 | 64% | 781.9446 | 782.3265 |

TABLE 4
First avian influenza candidate

| | | Theoretical | | Yield | | Percent | Molecular | |
|---|---|---|---|---|---|---|---|---|
| Name | Sequence | mg | μmol | mg | μmol | yield (%) | weight | Measured |
| Antigen | N₃-PEG-STQKAID | 391 | 400 | 218 | 223 | 56% | 977.027 | 978.1408 |
| peptide | | | | | | | | |
| MAP4 | Core-[PEG-STQKAID]₄ | 168 | 36 | 93 | 20 | 55% | 4690.053 | 4691.584 |

### Example 2

### <Procedure for Administration of Avian Influenza MAP4>

### Test:

Avian influenza MAP4 was dissolved in physiological saline containing 2% DMSO-1% mouse serum, and the resultant was administered to Balb/c mice by intraperitoneal injection. A solution of 100 μg of avian influenza MAP4 in 100 μl of physiological saline was administered to each Balb/c mouse in a single instance. MAP was administered once a day for 4 consecutive days (a total of 4 administration instances), and subsequent administration was performed at 7 days and at 14 days after the initial administration. The administration of α-galactosylceramide was carried out intraperitoneally at a dose of 2 μg/mouse together with MAP4 only in the initial administration. Blood was drawn from the orbital sinus of mice before administration and after administration, and the concentrations of anti-MAP antibodies in the sera were measured.

### <Method of Measurement of Antibody Titer of Avian Influenza MAP4 in Mouse Serum>

The anti-MAP antibody titer was measured in the manner described below. That is, bovine serum albumin (BSA) was bound to an avian influenza hemagglutinin peptide, the resultant was immobilized on ELISA plate, a 100-fold diluted serum was added thereto, incubation was carried out at 37° C. for 1 hour, the peroxidase-labeled anti-IgM antibody (SouthernBiotech), anti-IgG antibody (SouthernBiotech), anti-IgG1 antibody (SouthernBiotech), anti-IgG2a antibody (SouthernBiotech), or anti-IgG3 antibody (SouthernBiotech) was added thereto at the concentration recommended by the manufacturer, and color development (A 450) after the substrate was degraded by peroxidase was measured using a plate reader. Thus, the antibody titer in the serum was measured.

Also, bovine serum albumin (BSA) and FLAG were bound to an avian influenza hemagglutinin peptide, the resultant was immobilized on ELISA plate, the anti-FLAG monoclonal antibody (Clone: M2 mouse IgG, Sigma-Aldrich) diluted to various concentrations was added instead of the serum, measurement was carried out in the same manner as with the case of the anti-MAP antibody titer measurement, and a standard curve for quantification of the anti-MAP antibody titer was thus prepared. Based on the standard curve, an approximate concentration of the anti-MAP antibody in the serum was calculated.

### <Test Results>

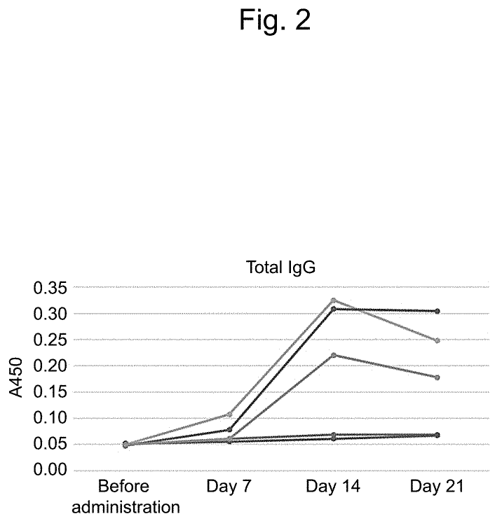

FIG. 2 shows the results of measurement of the anti-MAP antibody concentration in the serum.

As seen from FIG. 2, apparent increase in the IgG level was observed in 3 mice out of 5 mice to which the avian influenza MAP4 had been administered intraperitoneally (ip). The total levels of IgG antibody converted to anti-FLAG antibody were 157 ng/ml, 154 ng/ml, and 106 ng/ml, respectively, 14 days after administration, and it was 122 ng/ml, 148 ng/ml, and 83 ng/ml, respectively, 21 days after administration.

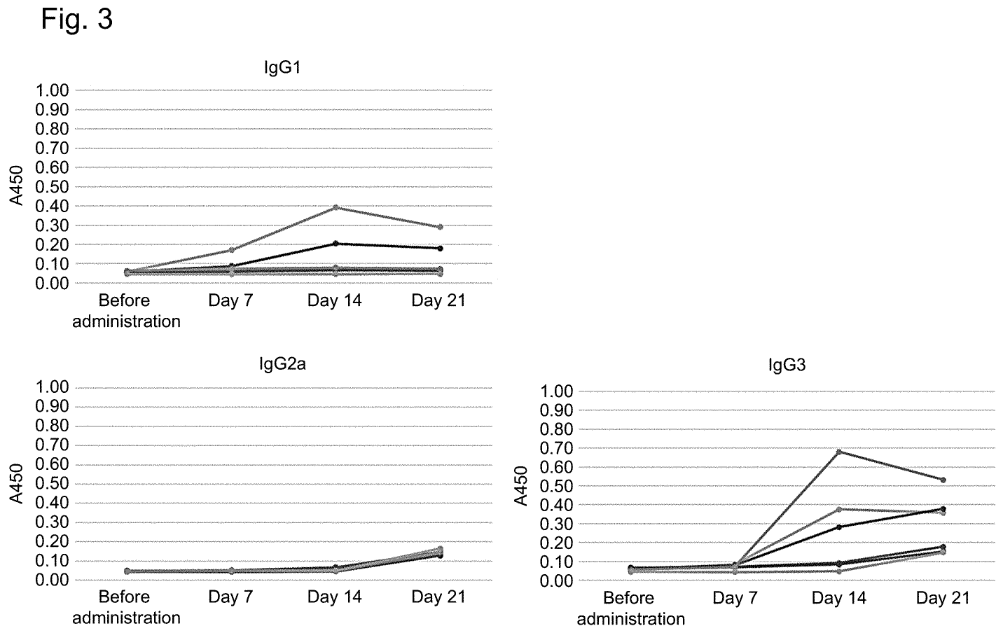

The subclasses of the resulting IgG antibodies were determined and, as a result, as shown in FIG. 3, subclasses exhibiting increased IgG antibody levels were found to be IgG and IgG3. Also, the increase in IgG1 level demonstrated that class switching of antibody genes had occurred.

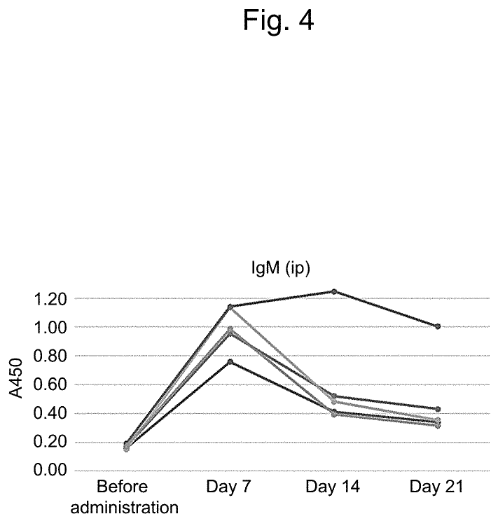

Moreover, as a result of measurement of the IgM titer, it was found that IgM antibodies were induced in all mice (5 out of 5 mice), as shown in FIG. 4.

Further when mice were subjected to the administration test described above by intravenous administration, similarly IgG and IgM antibodies against the avian influenza virus were found to be induced.

From the tests described above, the following results were attained.

(1) Intravenous or intraperitoneal administration of MAP4 and α-galactosylceramide enabled induction of IgG and IgM antibodies against the avian influenza virus.
(A) Intraperitoneal administration of α-galactosylceramide and the avian influenza MAP4 enabled induction of IgG and IgM antibodies against the avian influenza virus.
(B) The IgG concentration was slightly higher than 100 ng/ml.
(C) The subclasses of the IgG antibodies were IgG1 and IgG3.
(D) The IgM level increased in all mice after administration.
(2) It was found that the avian influenza MAP4 increases the antibody titer and has the function as a vaccine.
(3) Since IgM and IgG3 could efficiently have been induced, MAP4 was considered to act on B1B cells in the abdominal cavity.
(4) Since IgG1 was also induced, MAP4 was considered to partially act on B2B cells.

### INDUSTRIAL APPLICABILITY

The present invention provides an immunity inducer against avian influenza virus infection. As demonstrated in the examples, the multiple antigen peptide enabled the induction of IgG and IgM antibodies, indicating that the multiple antigen peptide could serve as a vaccine against the avian influenza virus. This is industrially useful in prevention and treatment of avian influenza virus infection.

All publications, patents, and patent applications cited herein are incorporated herein by reference in their entirety.

1. A multiple antigen peptide comprising a dendritic core and 4-8 antigen peptides, wherein each of the antigen peptides is bound to a terminus of the dendritic core directly or through a spacer, and is a peptide consisting of 7-12 consecutive amino acids in the amino acid sequence of SEQ ID NO: 4, or a peptide which is the same as the peptide except that 1-3 amino acids are substituted.

2. The multiple antigen peptide according to claim 1, wherein the peptide is a peptide consisting of 7-12 consecutive amino acids in the amino acid sequence of any of SEQ ID NOs: 5 to 9, or a peptide which is the same as the peptide except that 1-3 amino acids are substituted.

3. The multiple antigen peptide according to claim 1, wherein the peptide is a peptide consisting of 7-12 consecutive amino acids in the amino acid sequence of SEQ ID NO: 15, or a peptide which is the same as the peptide except that 1-3 amino acids are substituted.

4. The multiple antigen peptide according to claim 3, wherein the peptide is a peptide consisting of 7-12 consecutive amino acids in the amino acid sequence of any of SEQ ID NOs: 16 to 20, or a peptide which is the same as the peptide except that 1-3 amino acids are substituted.

5. The multiple antigen peptide according to claim 4, wherein the peptide is a peptide consisting of 7-12 consecutive amino acids in the amino acid sequence of any of SEQ ID NOs: 21 to 25, or a peptide which is the same as the peptide except that 1-3 amino acids are substituted.

6. The multiple antigen peptide according to claim 1, wherein all of the antigen peptides are peptides consisting of an identical amino acid sequence.

7. The multiple antigen peptide according to claim 1, wherein the dendritic core comprises a plurality of lysine residues.

8. The multiple antigen peptide according to claim 7, wherein the dendritic core further comprises a cysteine residue.

9. The multiple antigen peptide according to claim 1, wherein the spacer comprises a polyoxyalkylene chain.

10. The multiple antigen peptide according to claim 1, wherein the multiple antigen peptide is characterized by being represented by Formula (I): 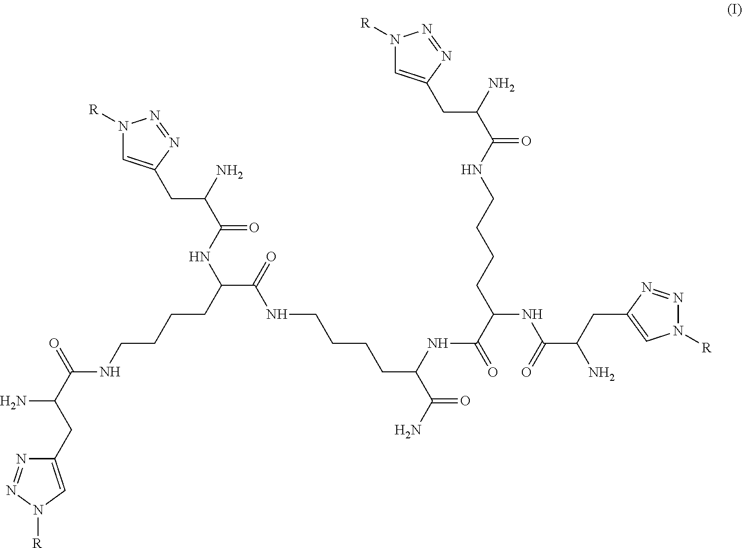
where R is: 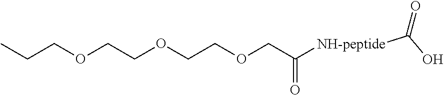
where the “peptide” represents an antigen peptide.

11. An immunity inducer comprising, one or at least two multiple antigen peptides according to claim 1.

12. The immunity inducer according to claim 11, which further comprises an adjuvant having an ability to produce interferon γ.

13. The immunity inducer according to claim 12, wherein the adjuvant is ca-galactosylceramide or an analog thereof.

14. The immunity inducer according to claim 11, which is used for treatment or prevention of avian influenza virus infection in a mammalian animal.

15. The immunity inducer according to claim 11, which further comprises a pharmaceutically acceptable carrier.

16. A method for treatment or prevention of avian influenza virus infection in a mammalian animal, comprising administering the immunity inducer according to claim 11 to the mammalian animal.


SEQUENCE LISTING (25 sequences)

SEQ ID NO: 1 - protein, 9 aa, Avian Influenza virus
GILGFVFTL

SEQ ID NO: 2 - protein, 9 aa, Avian Influenza virus
IILKANFSV

SEQ ID NO: 3 - protein, 9 aa, Avian Influenza virus
GMFNMLSTV

SEQ ID NO: 4 - protein, 21 aa, Avian Influenza virus
  misc_feature (1)..(1): Xaa is Gln or Glu
  misc_feature (3)..(3): Xaa is Ser, Thr, Glu or Val
  misc_feature (5)..(5): Xaa is Tyr, Gln, Thr or Met
  misc_feature (9)..(9): Xaa is Gln, Leu, Tyr or Arg
  misc_feature (10)..(10): Xaa is Lys, Glu or Asp
  misc_feature (14)..(14): Xaa is Asn, Ala, Lys or Ser
  misc_feature (18)..(18): Xaa is Gly, Gln or Lys
  misc_feature (19)..(19): Xaa is Ile or Val
  misc_feature (20)..(20): Xaa is Asn or Thr
  misc_feature (21)..(21): Xaa is Asn, Gly or Ser
XGXGXAADXXSTQXAIDXXXX

SEQ ID NO: 5 - protein, 21 aa, Avian Influenza virus
QGSGYAADQKSTQNAIDGITN

SEQ ID NO: 6 - protein, 21 aa, Avian Influenza virus
EGTGQAADLKSTQAAIDQING

SEQ ID NO: 7 - protein, 21 aa, Avian Influenza virus
QGSGYAADQESTQKAIDGVTN

SEQ ID NO: 8 - protein, 21 aa, Avian Influenza virus
QGEGTAADYKSTQSAIDQITG

SEQ ID NO: 9 - protein, 21 aa, Avian Influenza virus
QGVGMAADRDSTQKAIDKITS

SEQ ID NO: 10 - protein, 566 aa, Avian Influenza virus
  mat_peptide (345)..(566)
MEAKLFVLFCTFTVLKADTICVGYHANNSTDTVDTVLEKNVTVTHSVNLLEDSHNGKLCSLNGIAPLQLGKCNVAGWLLGNPECDLLLTANSWSYIIETSNSENGTCYPGEFIDYEELREQLSSVSSFEKFEIFPKTNSWPNHETTKGVTAACSYSGASSFYRNLLWITKKGTSYPKLSKSYTNNKGKEVLVLWGVHHPPTTSEQQSLYQNTDAYVSVGSSKYNRRFTPEIAARPKVRGQAGRMNYYWTLLDQGDTITFEATGNLIAPWYAFALNKGSDSGIITSDAPVHNCDTRCQTPHGALNSSLPFQNVHPITIGECPKYVKSTKLRMATGLRNVPSIQSRGLFGAIAGFIEGGWTGMIDGWYGYHHQNEQGSGYAADQKSTQNAIDGITNKVNSVIEKMNTQFTAMGKEFNNLERRIENLNKKVDDGFLDVWTYNAELLVLLENERTLDFHDSNVRNLYERVRSQLRNNAKELGNGCFEFYHKCDDECMESVKNGTYDYPKYSEESKLNREEIDGVKLESMGIYQILAIYSTVASSLVLLVSLGAISFWMCSNGSLQCRICI

SEQ ID NO: 11 - protein, 566 aa, Avian Influenza virus
  mat_peptide (346)..(566)
MKTIIVLSCFFCLAFSQNPSENNNNTATLCLGHHAVPNGTIVKTITDDQIEVTNATELVQSSSTGKICNNPHRILDGRDCTLMDALLGDPHCDVFQDETWDLYVERSSAFSNCYPYDVPDYASLRSLVASSGSLEFITEGFTWTGVTQNGGSGACKRGPANGFFSRLNWLTKSGNAYPLLNVTMPNNDDFDKLYVWGVHHPSTNQEQTSLYVQASGRVTVSTRRSQQTIIPNIGSRPWVRGQSGRISIYWTIVKPGDILVINSNGNLIAPRGYFKMRTGKSSIMGSDAPVDTCISECITPNGSIPNDKPFQNVNKITYGACPKYVKQSTLKLATGMRNVPEKQTRGLFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGCFKIYHKCDNACIESIRNGTYDHDIYRDEALNNRFQIKGVELKSGYKDWILWISFAISCFLLCVVLLGFIMWACQRGNIRCNICI

SEQ ID NO: 12 - protein, 568 aa, Avian Influenza virus
  mat_peptide (347)..(568)
MEKIVLLLATVSLVKSDQICIGYHANNSTEQVDTIMEKNVTVTHAQDILERTHNGKLCDLNGVKPLILRDCSVAGWLLGNPMCDEFINVPEWSYIVEKASPANDLCYPGNFNDYEELKHLLSRINHFEKIQIIPKSSWSNHDASSGVSSACPYLGRSSFFRNVVWLIKKNSAYPTIKRSYNNTNQEDLLVLWGIHHPNDAAEQTKLYQNPTTYISVGTSTLNQRLVPEIATRPEVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNTPQRERRRKKRGLFGAIAGFIEGGWQGMVDGWYGYHHSNEQGSGYAADQESTQKAIDGVTNKVNSIINKMNTPFEAVGREFNNLERRIENLNKKMEDGFLDVWTYNAELLVLMENERTLDFHDSNVKNLYDRVRLQLRDNAKELGNGCFEFYHKCDNECMESVKNGTYDYPQYSEEARLNREEISGVKLESMGTYQILSIYSTVASSLALAIMVAGLSLWMCSNGSLQCRICI

SEQ ID NO: 13 - protein, 560 aa, Avian Influenza virus
  mat_peptide (340)..(560)
MNTQILVFALIAIIPTNADKICLGHHAVSNGTKVNTLTERGVEVVNATETVERTNIPRICSKGKKTVDLGQCGLLGTITGPPQCDQFLEFSADLIIERREGSDVCYPGKFVNEEALRQILRKSGGIDKEAMGFTYSGIRTNGATSACRRSGSSFYAEMKWLLSNTDNAAFPQMTKSYKNTRKSPAIIVWGIHHSVSTAEQTKLYGSGNKLVTVGSSNYQQSFVPSPGARPQVNGLSGRIDFHWLMLNPNDTVTFSFNGAFIAPDRASFLRGKSMGIQSGVQVDADCEGDCYHSGGTIISNLPFQNIDSRAVGKCPRYVKQRSLLLATGMKNVPEIPKGRGLFGAIAGFIENGWEGLIDGWYGFRHQNAQGEGTAADYKSTQSAIDQITGKLNRLIEKTNQQFELIDNEFNEVEKQIGNVINWTRDSITEVWSYNAELLVAMENQHTIDLADSEMDKLYERVKRQLRENAEEDGTGCFEIFHKCDDDCMASIRNNTYDHSKYREEAMQNRIQIDPVKLSSGYKDVILWFSFGASCFILLAIVMGLVFICVKNGNMRCTICI

SEQ ID NO: 14 - protein, 560 aa, Avian Influenza virus
  mat_peptide (339)..(560)
METVSLITILLVATVSNADKICIGYQSTNSTETVDTLTENNVPVTHAKELLHTEHNGMLCATSLGQPLVLDTCTIEGLIYGNPSCDLSLEGREWSYIVERPSAVNGLCYPGNVENLEELRSLFSSARSYQRIQIFPDTIWNVSYDGTSSACSGSFYKSMRWLTRKNGDYPIQDAQYTNNQGKNILFMWGINHPPTDTTQRDLYTRTDTTTSVATEEINRIFKPLIGPRPLVNGLMGRIDYYWSVLKPGQTLRIKSDGNLIAPWYGHILSGESHGRILKTDLKRGSCTVQCQTEKGGLNTTLPFQNVSKYAFGNCSKYIGIKSLKLAVGLRNVPSRSSRGLFGAIAGFIEGGWSGLVAGWYGFQHSNDQGVGMAADRDSTQKAIDKITSKVNNIVDKMNKQYEIIDHEFSEVETRLNMINNKIDDQIQDIWAYNAELLVLLENQKTLDEHDANVNNLYNKVKRALGSNAVEDGKGCFELYHKCDDQCMETIRNGTYNRRKYQEESKLERQKIEGVKLESEGTYKILTIYSTVASSLVIAMGFAAFLFWAMSNGSCRCNICI

SEQ ID NO: 15 - protein, 12 aa, Avian Influenza virus
  misc_feature (4)..(4): Xaa is Gln, Leu, Tyr or Arg
  misc_feature (5)..(5): Xaa is Lys, Glu or Asp
  misc_feature (9)..(9): Xaa is Asn, Ala, Lys or Ser
AADXXSTQXAID

SEQ ID NO: 16 - protein, 12 aa, Avian Influenza virus
AADQKSTQNAID

SEQ ID NO: 17 - protein, 12 aa, Avian Influenza virus
AADLKSTQAAID

SEQ ID NO: 18 - protein, 12 aa, Avian Influenza virus
AADQESTQKAID

SEQ ID NO: 19 - protein, 11 aa, Avian Influenza virus
AADKSTQSAID

SEQ ID NO: 20 - protein, 12 aa, Avian Influenza virus
AADRDSTQKAID

SEQ ID NO: 21 - protein, 7 aa, Avian Influenza virus
  misc_feature (4)..(4): Xaa is Asn, Ala, Lys or Ser
STQXAID

SEQ ID NO: 22 - protein, 7 aa, Avian Influenza virus
STQNAID

SEQ ID NO: 23 - protein, 7 aa, Avian Influenza virus
STQAAID

SEQ ID NO: 24 - protein, 7 aa, Avian Influenza virus
STQKAID

SEQ ID NO: 25 - protein, 7 aa, Avian Influenza virus
STQSAID